(19) United States
(12) Patent Application Publication
(10) Pub. No.: US 2023/0088240 A1
(43) Pub. Date: Mar. 23, 2023

(54) ACCELERATING THE DRYING RATE OF SOFTGEL CAPSULES

(71) Applicants: Diego Rafael Monterroza Hernández, Barranquilla (CO); Claudia Andrea Silva Blanco, Barranquilla (CO)

(72) Inventors: Diego Rafael Monterroza Hernández, Barranquilla (CO); Claudia Andrea Silva Blanco, Barranquilla (CO)

(21) Appl. No.: 18/051,494

(22) Filed: Oct. 31, 2022

Related U.S. Application Data
(63) Continuation-in-part of application No. 17/706,766, filed on Mar. 29, 2022, now abandoned.
Application No. 17/706,766 is a continuation-in-part of application No. 17/412,515, filed on Aug. 26, 2021, now abandoned.
Application No. 17/412,515 is a continuation-in-part of application No. 17/149,644, filed on Jan. 14, 2021, now abandoned.
Application No. 17/149,644 is a continuation-in-part of application No. 16/894,787, filed on Jun. 6, 2020, now abandoned.
Application No. 16/894,787 is a continuation-in-part of application No. 16/658,616, filed on Oct. 21, 2019, now abandoned.
Application No. 16/658,616 is a continuation-in-part of application No. 16/368,132, filed on Mar. 28, 2019, now abandoned.
Application No. 16/368,132 is a continuation-in-part of application No. 16/104,931, filed on Aug. 19, 2018, now abandoned.
(60) Provisional application No. 62/437,093, filed on Dec. 21, 2016.

(51) Int. Cl. A61K 9/48; A61K 31/63; A61K 33/12; A61K 9/16; A61K 31/255

(52) U.S. Cl. CPC A61K 9/4858; A61K 31/63; A61K 33/12; A61K 9/1629; A61K 31/255

(57) ABSTRACT

The instant invention provides a process for reducing the drying time of softgel capsules by incorporating drying accelerators based on organic sulfonic acids and salts thereof into the formulations used to make the capsules.

This application is a continuation-in-part of U.S. Ser. No. 17/706,766 filed Mar. 29, 2022, which application is a continuation U.S. Ser. No. 17/412,515 filed Aug. 26, 2021; which application is a continuation U.S. Ser. No. 17/149,644 filed Jan. 14, 2021; which application is a continuation-in-part of U.S. Ser. No. 16/894,787 filed Jun. 6, 2020; which application is a continuation-in-part of U.S. Ser. No. 16/658,616 filed Oct. 21, 2019; which application is a continuation-in-part of U.S. Ser. No. 16/368,132 filed Mar. 28, 2019; which application is a continuation-in-part of U.S. Ser. No. 16/104,931 filed Aug. 19, 2018; which application is also a continuation-in-part of U.S. Ser. No. 15/851,683 filed Dec. 21, 2017. This application also claims the priority benefit under 35 U.S.C. section 119 of U.S. Provisional Patent Application No. 62/437,093 entitled “Accelerating The Drying Rate Of Softgel Capsules” filed on Dec. 21, 2016; which is in its entirety herein incorporated by reference.

### FIELD OF THE INVENTION

This invention relates to stable soft gel capsules made from gelatin or other materials suitable for making soft capsules (hereinafter softgel) containing pharmaceutically active ingredients and to a process for the preparation of therapeutically useful, highly stable, soft gelatine capsules. The present invention also relates to soft gelatin capsules with a gelatin shell, at least one plasticizer, a capsule additive that enhances the drying rate of said capsule and a capsule filling which contains at least one pharmacologically-active substance and a solvent[DMH1], as well as processes for their manufacture.

The present invention further relates to a process for accelerating the drying rate of softgel capsules by incorporating an additive in the softgel formulation that enhances the drying rate of the capsules.

### BACKGROUND OF THE INVENTION AND DESCRIPTION OF THE PRIOR ART

Capsules are solid dosage forms in which fill materials are enclosed in a gel wall. The fill materials may include paintball fill materials and therapeutic agents, and at least in the case of the latter, the gel wall of prior art capsules has typically been formed as a soluble gelatin wall. The wall of a gelatin capsule can be made of either soft or hard gelatin. In the prior art, the gelatin may in some cases have been plasticized by the addition of additives such as glycerin, sorbitol, and/or similar types of polyols. Soft gelatin capsules have offered a convenient dosage form for the administration of drugs, nutrients, vitamins, foodstuff and cosmetics. Commercially available gelatin capsules have been provided in a variety of sizes and shapes, e.g., round, oval, oblong, tubular, and suppository-shaped.

It is well known that liquid, and especially concentrated liquid pharmaceutical compositions offer many advantages over solid compositions. Liquids are easy to swallow and provide an excellent vehicle for the uniform delivery of pharmaceutical actives. Liquids provide a rapid onset of pharmacologic action, since the composition does not first have to disintegrate and dissolve in the gastrointestinal tract. Concentrated liquid compositions are ideally suited for encapsulation within a soft gelatin shell, to provide a portable and easy-to-swallow soft, flexible capsule. Encapsulation would also permit the accurate and uniform delivery of a unit dose of a pharmaceutical active, an advantage which becomes especially important when relatively small amounts of an active are to be delivered. Additionally, soft gelatin capsules are aesthetically appealed (especially when filled with a transparent liquid) and can be manufactured in a wide variety of sizes, shapes, and colors.

Gelatin capsules, especially soft gelatin capsules, have become increasingly important as a medical dosage form since it became feasible, in the 1930's, to manufacture them by making and filling the capsules in one operation. Compared to other medical dosage forms they show many advantages such as being odorless and tasteless, they can be taken easily and, owing to their swelling capability and water solubility, the drugs are readily liberated in the stomach. Numerous drugs which, on account of their instability such as sensitivity to oxidation and to light, their thermal stability or their hygroscopicity, may not be easily processed into other medicinal forms, can be encapsulated without impairment of their function.

Softgels provide several advantages such as easy to swallow, no taste, unit dose delivery, tamper-proof, wide variety of colors, shapes, and sizes, accommodates a wide variety of compounds filled as a semi-solid, liquid, gel or paste, immediate or delayed drug delivery and can be used to improve bioavailability by delivering drug in solution or other absorption enhancing media.

The need for encapsulation of pharmaceutically active dosage forms such as liquids, semi-solids, pastes, and solids within a gelatin shell in such a way as to prevent uncontrolled leakage has resulted in the development of a very fundamental pharmaceutical dosage form: the soft gelatin capsule. While a difficult and not particularly accurate process initially, current manufacturing processes for softgels are fully automated, with a high degree of precision.

The softgel (the currently accepted nomenclature adopted by the SoftGel Association) is a one-piece, hermetically sealed soft gelatin shell containing a liquid, a suspension, or a semi-solid.

The most common modern production method involved in the preparation of softgels is a continuous method whereby two gelatin ribbons pass between twin rotating dies. As the ribbons meet, the liquid to be encapsulated is precisely injected between them. The capsule halves are sealed and ejected by the continuous rotation of the dies. See P. Tyle, Specialized Drug Delivery Systems, Marcel Dekker, Inc. (1990) for a general discussion of softgel manufacturing and production technology, in particular, Chapter 10 by Paul K. Wilkinson and Foo Song Hom.

Various gelatin shell masses may be prepared, depending on the fill properties, climatic conditions, and end use. Typically, gelatin formulations include the same basic ingredients, namely, gelatin, plasticizers such as glycerin, water, and optionally preservatives as well as other optimizing ingredients. The formulations of gelatins are well known to those of ordinary skill in the art.

The process for making softgel capsules includes the step wherein the gelatin shell and the fill material come together to form Softgel capsules. It takes place in a closed environment called clean room where the relative humidity is around 20%. The gelatin shell and fill material are brought together simultaneously in the encapsulation machine.

The process is basically performed as follows: a pump delivers the warm gelatin over two chilled drums which are located at both opposite sides of the machine, through a spreader box that sits over each drum. The warm liquid gelatin flows over the drums and this transforms the liquid gelatin into two solid ribbons of gel. The left and right ribbons pass over rollers which feed them through two die rolls. These die rolls determine the shape and size of softgels and cut the Softgel shell from the ribbons as they turn around.

Simultaneously, a sensitive and high accuracy positive displacement pump delivers the fill material into a heated wedge which sits between rotary dies. This wedge injects the fill material into the die cavities between ribbons just right before the die rolls cut the ribbons and seal the two halves together. Warm just formed softgels slide gently through a chute onto a conveyor belt which carries them to the tumble dryer where cooling and drying process takes place.

In more specific detail, typical soft encapsulation machines form at least two flexible gelatin sheets or ribbons by cooling molten gelatin on separate drums then lubricating and guiding the sheets into communication with each other over co-acting dies while simultaneously dispensing a desired quantity of fill material between the sheets in synch with cavities in the outer surfaces of the dies to produce soft capsules. The encapsulation machines typically utilize gearing to control the relative rotations of the various components and fill mechanisms to synchronize the operation of these various components. The synchronization of these various components, however, can vary depending upon a variety of factors, such as the particular dies used, the number of cavities and the size of the cavities on the dies, and the type of material used to form the sheets. To change the synchronization of the various components, mechanical gears are required to be changed to obtain the desired ratios and synchronization of these components. The changing of gears, however, is time intensive. Additionally, the use of mechanical gears provides finite gear ratios which limit the synchronization of the various components to the mechanical gears that are available. Thus, it would be advantageous to provide a capsule machine wherein the synchronization and rates at which the various components operate can be altered without the necessity of changing gears. Additionally, it would be advantageous if the synchronization between the various components can be infinite to thereby allow more precise synchronization between the various components. It would also be advantageous to allow various components, such as the fill mechanism, to be adjusted independently of the other components while the machine is running to allow for adjustments of the timing of fill material inserted into each of the soft capsules. It would also be advantageous to eliminate the use of casting drums in the making of softgel capsules.

During the operation of the capsule making machine, the contact between the adjacent dies can be adjusted by the operator of the capsule making machine. Typically, the operator is able to move one of the dies closer to the other die so that the pressure or force exerted on the sheets passing between the adjacent dies can be adjusted. Such adjustments, typically are mechanical adjustments made by fluid actuators, such as pneumatic cylinders. The operator is able to adjust the pneumatic pressure thereby altering the force the dies exert on one another and on the sheets. This adjustability allows an operator to customize the pressure to ensure that quality soft capsules are produced. However, the dies are susceptible to premature failure and/or wear when the pressure or force between the two dies is more than that required to produce acceptable soft capsules. Thus, it would be advantageous to monitor/record the pressure applied to the dies so that quality capsules are produced without inducing excessive wear or premature wear on the dies.

A material fill mechanism is used to supply the fill material that is encapsulated within the soft capsules. When the fill material is a liquid, such as a liquid medication or die for a paint ball capsule, the fill mechanism includes a plurality of positive displacement plunger-type pumps that are arranged in a housing above the dies. The plunger-type pumps are positioned on a yoke that moves linearly in a reciprocating motion to allow the plunger-type pump to fill with the liquid fill material on one stroke and subsequently discharge the liquid fill material on the other stroke. A valving arrangement between opposing pumps is utilized to control the discharge and filling of the pumps. The valve arrangement includes a sliding member that moves linearly back and forth in a direction generally perpendicular to the linear motion of the yoke. The discharge of the liquid fill material into the sheets as they are passing through the dies is coordinated with the operation of the dies to insure that the timing of the injection of the liquid fill material is synchronized with the cavities on the dies. Typically, this synchronization has been performed through the use of mechanical gears that link the timing of the stroke to the rotation of the dies. Thus, in order to adjust the synchronization a mechanical gear change is required which is time consuming. Additionally, the timing is limited to a finite number of gear ratios as determined by the gears that are available.

The sliding member of the valving mechanism requires lubrication. Typically, the lubrication is provided by a lubricating pump with its own separate drive. However, the use of a separate drive to operate the lubricating pump adds additional complexity and components to the capsule machine. Thus, it would be advantageous if a motion of the slide member and/or the yoke could be utilized to drive the lubrication pump.

The pumps are typically contained within a housing that is filled with a lubricating oil that is used to lubricate the sliding member. The pumps, however, can leak around their seals and contaminate the lubricating oil with the leaking fill material. Contamination of the oil requires a time consuming and possibly difficult clean up and can cause the lubricating oil to not perform as designed thereby increasing the wear on the sliding surfaces and decreasing the life span of the sliding surfaces. Thus, it would be advantageous to capture any fill material that leaks from the pumps and deter or prevent the liquid fill material from contaminating the lubricating oil within the pump housing.

The pumps are typically driven by a drive mechanism that is also located within the pump housing. Because the drive mechanism is located in the pump housing, it is possible for liquid fill material that leaks from the pumps to contaminate not only the lubrication oil but also the drive mechanism. When switching from one fill material to another, the pump and all of the components in the pump housing are required to be thoroughly cleaned to remove all contamination. The locating of the drive mechanism within the pump housing provides additional components that must also be cleaned when changing the fill material. Thus, it would be advantageous to separate the drive mechanism from the pump housing to reduce the components that are required to be cleaned when changing fill material.

In most cases, the typical rotary die process requires a flowable liquid or fill containing a bio-active ingredient. The fill may be a single phase liquid active, a mixture of miscible liquids, or a solution or a suspension of solids and liquids as well as powders, microgranules, nanogranules and solids. Generally, the fill contains glycerin and a medicament. The liquids to be encapsulated in a gelatin shell are also well known to those of ordinary skill in the art.

Many shell and fill formulations are discussed in Van Hostetler and J. Q. Bellard noted below as well as in “Advances in Softgel Formulation Technology”, M. S. Patel, F. S. S. Morton and H. Seager, Manufacturing Chemists, July 1989; “Soft Elastic Gelatin Capsules: A Unique Dosage Form”, William R. Ebert, Pharmaceutical Technology, October 1977; “Soft gelatin capsules: a solution to many tableting problems”, H. Seager, Pharmaceutical Technology, September 1985; U.S. Pat. No. 4,067,960 to Fadda; U.S. Pat. No. 4,198,391 to Grainger; U.S. Pat. No. 4,744,988 to Brox; and U.S. Pat. No. 4,780,316 to Brox. All of the above references are incorporated herein by reference.

Subsequent to the rotary die process used to produce the gelatin shells having a medicament fill therein, the resulting capsules are typically washed with a solvent that evaporates easily. Thereafter, the capsules are typically tumble dried in a series of hollow drums with perforated walls. Heated dry air is continuously pumped through the rotating drums at an air temperature typically less than 35° C. The warm air blown into the capsules appears to penetrate the shell and cause it to dry from the inside by moving the water outward to the surface of the capsule. By the time the capsules exit this process, all of the solvent used in washing has typically been evaporated, and a large proportion (50-60%) of the water from the gelatin shell has been removed. Recent developments in drying include bypassing the drum drying stage and having the capsules dried in a drying tunnel or room as further discussed below.

After the capsules exit the last drying drum, the capsules are typically spread on drying trays. The final drying phase for softgels is typically accomplished by passing the drying trays through drying tunnels or into drying rooms. Stacks of trays are inserted into drying tunnels or drying rooms, in which controlled temperature air (21°−24° C.) and low relative humidity (20-30%) is continuously circulated. Although additional water may be removed from dry capsules by further heating, for example at 40° C., such a procedure has not been found to be practical or necessary. See Van Hostetler and J. Q. Bellard in The Theory and Practice of Industrial Pharmacy, “Capsules”, (1970), Chapter 13 at pages 346-383, and in particular at page 380.

The drying time, for most softgels, is 16-24 hours, but may be slightly longer if the softgels are over 20 minims in size or if the softgels contain a non-oily type liquid base. Softgels permitted to come to water equilibrium in this controlled environment are considered “dry”. The gelatin fill and shell of such “dry” softgels contain 6-10% water depending on the specific gelatin and fill formula used. After drying, the capsules are typically inspected and finished using varied known techniques.

Additionally, soft gelatin capsules containing hydrophilic fill contents experience on drying migration between the fill content and the gelatin shell. This mass transfer depends on type and levels of plasticisers in the shell, on the fill composition (cosolvents and water content) as well as on the room conditions (temperature and relative humidity). As result of migration some products could dry as long as 5-7 days at 18° C./25% RH. Equilibrium at drying is evaluated in the development of the product in a case to case base and the criteria is the fill content moisture (9-12%) since lower moisture values could lead to crystallization of the API and higher moisture values could lead to softening or microbial growth.

Commercial sodium docusate soft gelatin capsules containing sodium docusate in the fill content have shown migration of SD to the shell dependent on the SD concentration, the higher the SD concentration in the product, the higher the amount of SD that migrates to the shell. In addition, SD hydrophilic SGC get dried in 2-3 days, a shorter time compared to other hydrophilic SGC similar in composition and capsule size that usually take at least 5 days to get dried.

The present invention seeks to accelerate the drying rate of softgel capsules whether they are tumble dried or dried in trays.

### OBJECTS OF THE INVENTION

It is an object of the present invention to provide new softgel formulations containing additives that enhance the drying rate of the capsules.

It is also an object of the present invention to provide a process for accelerating the drying rate of softgel capsules.

It is a specific object of the present invention to provide softgel capsules incorporating an accelerator for drying.

It is a further object of the invention to use organic sulfonic acids and salts thereof as accelerators for drying softgel capsules.

This invention also has for its object a method for the drying of soft gelatin capsules in a particularly expeditious and economical manner and, at the same time provides a method of insuring that they are evenly formed and fault-free.

Other objects and embodiments of the present invention will be discussed below.

However, it is important to note that many additional embodiments of the present invention not described in this specification may nevertheless fall within the spirit and scope of the present invention and/or the claims.

### BRIEF DESCRIPTION OF THE FIGURES

FIG. 1 shows the apparatus to make the capsules of the invention.

FIG. 2 illustrates the drying curves for soft gel capsules (SGC) with added sodium docusate SD at different S/G levels.

FIG. 3 describes the hardness of soft gel capsules (SGC) with added SD at different S/G levels.

FIG. 4 shows the drying curves soft gel capsules (SGC) at different added sodium docusate (SD) concentrations (S/G 1.5).

FIG. 5 illustrates the hardness of soft gel capsules (SGC) at different SD concentrations (S/G 1.5) and drying times.

FIG. 6 is a front view of the complete apparatus of the invention showing all the elements of the apparatus.

FIG. 7 is also a front view of the apparatus of FIG. 1 without the spreader boxes and casting drums.

FIG. 8 is a front view of the mechanism for filling the capsules with other capsules.

FIG. 9 is also a front view of how the smaller capsules are dispensed into the larger capsule.

FIG. 10 shows the smaller capsule hopper having capsules which are fed via guiding channels into the larger capsule.

FIG. 11 shows a representative end product of the invention containing two capsules inside another capsule wherein the larger capsule incorporates a sulfonate additive.

FIG. 12 are representative examples of products contemplated by the invention similar to the ones in FIG. 6.

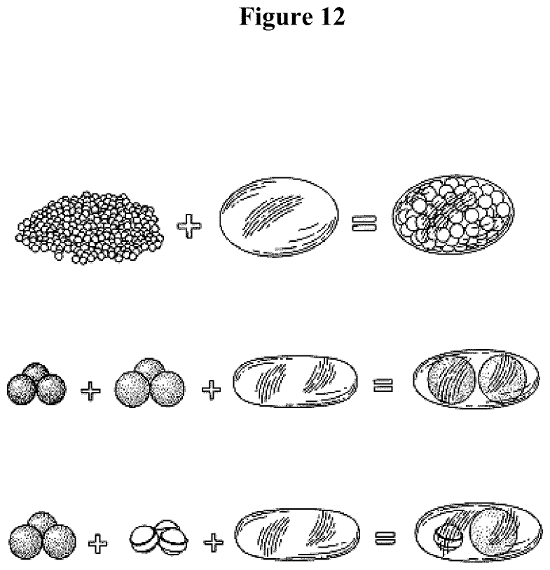

### SUMMARY OF THE INVENTION

The invention provides a softgel capsule having incorporated in the shell an amount of an organic sulfonate salt which is effective to accelerate the drying rate of said capsule but not effective to provide a pharmacological effect.

The present invention also relates to the use of organic sulfonate salts to accelerate the drying of softgel capsules.

The invention further features accelerating the drying rate of soft gelatin capsules by incorporating in the gelatin formulation an ester of an aliphatic dibasic acid having the formula

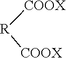

in which R is an aliphatic carbon chain containing at least one sulphonic group but free from other substituents, and X is hydrogen or an alcohol or phenol radical not connected by a carbon to carbon bond with R, at least one X being such an alcohol or phenol radical.

The invention further relates to accelerating the drying rate of soft gelatin capsules by incorporating in the gelatin formulation an ester of an aliphatic dibasic acid having the formula

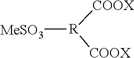

in which R is an aliphatic carbon chain containing at least one sulphonic group but free from other substituents, and X is hydrogen or an alcohol or phenol radical not connected by a carbon to carbon bond with R, at least one X being such an alcohol or phenol radical and Me is hydrogen or a base.

The invention also describes accelerating the drying rate of soft gelatin capsules by incorporating in the gelatin formulation an ester of an aliphatic dibasic acid having the formula

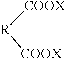

in which R is an aliphatic carbon chain containing at least one sulphonic group, but free from other substituents, and X is an alcohol or phenol radical not connected by a carbon to carbon bond with R.

The invention further relates to accelerating the drying rate of soft gelatin capsules by incorporating in the gelatin formulation an ester of an aliphatic dibasic acid having the formula

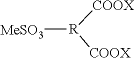

in which R is an aliphatic carbon chain containing at least one suiphonic group, but free from other substituents, X is an alcohol or phenol radical not connected by a carbon to carbon bond with R, and Me is hydrogen or a base.

The invention is also directed to accelerating the drying rate of soft gelatin capsules by incorporating in the gelatin formulation an ester of an aliphatic dibasic acid having the formula

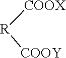

in which R is an aliphatic carbon chain containing at least one sulphonic group and free from mercapto groups and X is an alcohol or phenol radical and Y is a different alcohol or phenol radical.

The invention further describes accelerating the drying rate of soft gelatin capsules by incorporating in the gelatin formulation an ester of an aliphatic dicarboxylic acid having the following formula

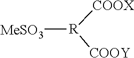

MeSO₃—R in which R is a carbon chain free from mercapto groups, Me is a base, X is an alcohol or phenol radical and Y is a different alcohol or phenol radical.

The present invention provides gelatin formulations incorporating organic sulfonate salts additives which reduce the drying time of soft gelatine capsules. The additive may also e included in the fill formulation that goes inside the shells. Additionally, the invention is intended to include capsules made from alternate sources of gelatin such as cellulosic materials, gums, vegetable sources of equivalent materials that are similar to gelatin.

The present invention provides gelatin formulations incorporating organic sulfonate salts additives which reduce the drying time of soft gelatine capsules. The additive may also e included in the fill formulation that goes inside the shells. Additionally, the invention is intended to include capsules made from alternate sources of gelatin such as cellulosic materials, gums, vegetable sources of equivalent materials that are similar to gelatin.

The instant invention also provides a process for reducing the drying time of softgel capsules by incorporating drying accelerators based on organic sulfonic acids and salts thereof into the formulations used to make the capsules. It should be noted that the invention includes hydrophilic, lipophilics, self-emulsifying, solutions and suspensions as the fill contents.

The softgel capsules are evaluated for physical and chemical stability under accelerated conditions based on the capsules obtained with the selected gelatin formulations.

By way of background, it is known that the drying time of the sodium docusate and calcium docusate CBG product (as an active pharmaceutical in the capsule) is 24-48 h, compared to similar hydrophilic formulations with drying times in some cases up to 144 h.

It is known that some surfactants have the property of acting as permeation enhancers of polymer films, favoring the mobilization of water and increasing the speed of drying.

In the process of manufacturing soft gelatin capsules, drying is the process step with longer processing times and costs associated with time and energy consumption. For this reason, an effective measure in the reduction of the drying times will represent benefits in the reduction of the manufacturing costs, as well as in the optimization of this stage of manufacture and in the increase of the productive capacity of the manufacturing plant.

In the method of the invention, the amount of organic sulfonate salt is between 0.1 mg to about 20 mg. The amount used is sufficient to enhance the drying rate of the softgel capsule.

The present invention is also directed to the use of sodium docusate as an accelerator of drying of softgel capsules. The docusate is present in amounts to accelerate the drying rate but not in amounts to exert a pharmacological effect.

The present invention also responds specifically to the long-felt need heretofore unmet by the prior art, and especially with a view to overcoming the inherent inadequacies of combination of pharmaceuticals that are not compatible for oral delivery to mammals. The composition is a pharmaceutical combination i.e., a capsule within a capsule providing the convenience and reliability of oral administration, while providing near simultaneous delivery in vivo of incompatible substances. The composition is shelf stable when formulated and it includes a sulfonate additive to accelerate the drying rate of the soft gel capsules.

The foregoing, and other advantages of the present invention, are realized in one aspect thereof in an oral pharmaceutical composition that is a combination of incompatible active ingredients. The composition comprises a double soft capsule which includes one pharmaceutical in a first capsule which is enclosed in a second soft capsule also containing a second active ingredient. The soft capsules are preferably made of gelatin wherein the shell composition includes an organic sulfonate to accelerate the drying of the capsules. The active ingredients may be combined with acceptable grade carriers.

In another aspect, the invention is a method of simultaneously delivering incompatible compounds to mammals in vivo. Such delivery is achieved by administering orally to a mammal a double soft capsule containing a first substance in a first capsule, which is enclosed with a second substance, incompatible with the first substance, in a second larger soft capsule which outer shell includes an organic sulfonate additive that accelerates the drying of said capsules.

In another embodiment, this invention provides a method for preparing shelf-stable compositions of incompatible substances, which includes the use of multiple capsules of variable composition. Such method is accomplished manually or by the apparatus of the invention further described below.

As used herein, the term “incompatible” is meant to refer to substances which deleteriously react with one another when combined in desired levels or concentrations.

The invention also provides an apparatus for making softgel capsules having incorporated therein other solid dosage forms selected from the group consisting of pellets, smaller capsules, smaller tablets, sustained release solid dosage forms, immediate release solid dosage forms, extended release solid dosage forms and zero order release solid dosage forms, said apparatus comprising: (a) two spreader boxes; (b) two casting drums; (c) a pair of rotary dies having means for suction; (d) a liquid fill system; (e) a wedge for heating gelatine ribbons and feeding said fill; and (f) two lateral dispensing devices said lateral dispensing devices including hoppers having said solid dosage forms, channel guides for transporting said solid dosage forms and a grasping claw for dispensing said solid dosage form into the softgel pocket formed in the rotary dies.

The invention further provides a dispensing device for dispensing and feeding solid dosage forms into a softgel capsule said dispensing and feeding device including a hopper having said solid dosage forms, channel guides for transporting said solid dosage forms and a grasping claw for dispensing said solid dosage form.

The instant invention also provides a method for making softgel capsules having incorporated therein other solid dosage forms, said method comprising: casting a gel forming composition to make films; (b) passing said films through a pair of rotary dies having vacuum means to make pockets; (c) feeding smaller solid dosage forms into said pockets using a lateral dispensing and feeding system that uses a grasping claw; (d) filling said pockets with a medicine formulation in liquid form via a wedge segment; and (e) forming said capsule by sealing the pockets together wherein said capsules include in the shell composition an organic sulfonate to accelerate the drying rate of said capsules.

The invention is also a process for making a softgel capsule having incorporated therein another capsule, said process comprising: (a) feeding film sheets between a first die roll and a second die roll wherein each of the die rolls have capsule pockets in a plurality of rows and said capsule pockets have at least one orifice for application of suction; (b) applying suction while said film is in place in the capsule pockets; (c) feeding via guide channels through a lateral dispensing device having a hopper and a grasping claw preformed smaller capsules onto the film sheets overlying the die rolls at positions having the capsule pockets; (d) filling said capsule pockets also via a wedge segment with a liquid medical formulation; and (e) cutting the film sheets about the capsule pockets to form said soft gel capsules having capsules in combination with a suitable liquid pharmaceutical combination.

The invention further provides softgel capsules incorporated into an outer softgel capsule, and tablets or other solid forms incorporated into an outer softgel capsule, microgranules incorporated into an outer softgel capsule, and any combination between softgels, tablets and/or microgranules incorporated into an outer softgel capsule.

The instant invention also provides a softgel capsule having incorporated therein another solid dosage form selected from the group consisting: (a) one capsule contains an omega oil and the other solid dosage form is a capsule having a statin; (b) one capsule contains a non-steroidal antiinflammatory and the other solid dosage form contains and antihistamine; and (c) one capsule contains and omega oil and the other solid dosage form contains a salicylate and wherein the capsule making composition includes a an organic sulfonate to accelerate the drying rate of the resulting capsules.

Other advantages and a fuller appreciation of the specific adaptations, compositional variations, and physical and chemical attributes of the present invention will be gained upon an examination of the following detailed description of the invention, taken in conjunction with the accompanying drawings and appended claims.

### DETAILED DESCRIPTION OF THE INVENTION

The soft gelatin shells of the instant invention can be prepared by combining appropriate amounts of gelatin, water, plasticizer, drying accelerant and any optional components in a suitable vessel and agitating and/or stirring while heating to about 65° C. until a uniform solution is obtained. This soft gelatin shell preparation can then be used for encapsulating the desired quantity of the solubilized fill composition employing standard encapsulation methodology to produce one-piece, hermetically-sealed, soft gelatin capsules.

In a nut shell, the formation of soft gelatin capsules is carried out in a stamping process wherein the capsule wall is assembled from two gelatin halves which are stamped out of a gelatin band and then molded. Preferably, there is utilized the rotary die process operating under the rotary die method. Herein two endless gelatin bands run against two adjacent and mutually counter-rotating molding rollers. While the gelatin bands are being pressed into the molded and so create the capsule halves, the flowable filler is provided into the thus formed capsule through an exact dosing wedge. There follows the sealing together of the capsule halves, their stamping out, a wash procedure for the freeing of attached oil, a rotational dryer step as well as an adjacent shelf drying.

More specifically, the fill formulation of the instant invention is encapsulated into one-piece gelatin sheath or shell that includes a plasticizer to control the softness and flexibility of the sheath, water, and optionally, other additives, such as flavorants, colorants, opacifiers, etc. The softgel capsules may be produced in a known manner with a rotary die process in which a molten mass of a gelatin sheath formulation is fed from a reservoir onto drums to form two spaced sheets or ribbons of gelatin in a semi-molten state. These ribbons are fed around rollers and brought together at a convergent angle into the nip of a pair of roller dies that include opposed die cavities. A fill formulation to be encapsulated is fed into the wedge-shaped joinder of the ribbons.

The gelatin ribbons are continuously conveyed between the dies, with portions of the fill formulation being trapped between the sheets inside the die cavities. The sheets are then pressed together, and severed around each die so that opposed edges of the sheets flow together to form a continuous gelatin sheath around the entrapped medicament. The part of the gelatin sheet that is severed from the segments forming the capsules is then collected for recycling, and the soft capsules are dried.

Various sheath formulations known in the prior art may be used to encapsulate the fill formulations of the present invention. For example, suitable sheath formulations may include from about 30 to about 50% by weight gelatin; at least 18% by weight, and preferably up to about 40% by weight, of a plasticizer; and from about 20 to about 50% by weight water. These formulations, when formed into capsules and dried, will result in capsule sheaths comprised of from about 40 to about 75% by weight gelatin; from about 18% to about 40% by weight plasticizer; and from about 5 to about 15% by weight water.

The gelatin will normally have a bloom in the range of from about 140 to about 280, and may be Type A or B gelatins or a mixture thereof. Limed bone, acid bone, fish and/or pig skin gelatins may be used.

The gelatin capsules are formed into the desired shape and size so that they can be readily swallowed. The soft gelatin capsules of the instant invention are of a suitable size for easy swallowing and typically contain from about 100 mg to about 2000 mg of the solubilized pharmaceutical active composition. Soft gelatin capsules and encapsulation methods are described in P. K. Wilkinson et al., “Softgels: Manufacturing Considerations”, Drugs and the Pharmaceutical Sciences, 41 (Specialized Drug Delivery Systems), P. Tyle, Ed. (Marcel Dekker, Inc., New York, 1990) pp. 409-449; F. S. Hom et al., “Capsules, Soft” Encyclopedia of Pharmaceutical Technology, vol. 2, J. Swarbrick and J. C. Boylan, eds. (Marcel Dekker, Inc., New York, 1990) pp. 269-284; M. S. Patel et al., “Advances in Softgel Formulation Technology”, Manufacturing Chemist, vol. 60, no. 7, pp. 26-28 (July 1989); M. S. Patel et al., “Softgel Technology”, Manufacturing Chemist, vol. 60, no. 8, pp. 47-49 (August 1989); R. F. Emerson, “Softgel (Soft Gelatin Capsule) Update”, Drug Development and Industrial Pharmacy (Interphex '86 Conference), vol. 12, no. 8 & 9, pp. 1133-1144 (1986); and W. R. Ebert, “Soft Elastic Gelatin Capsules: A Unique Dosage Form”, Pharmaceutical Technology, vol. 1, no. 5, pp. 44-50 (1977); these references are incorporated by reference herein in their entirety. The resulting soft gelatin capsule is soluble in water and in gastrointestinal fluids. Upon swallowing the capsule, the gelatin shell rapidly dissolves or ruptures in the gastrointestinal tract thereby introducing the pharmaceutical actives into the physiological system.

The particular sulfonate salts used in the invention include metal sulfonates derived from Benzenesulfonate, Camphorsulfonate, Sterylsulfate, 4-Chlorobenzenesulfonate, 6,7-Dihydroxycoumarin-4-methanesulfonate, 1,2-Ethanedisulfonate Edisylate, Lauryl sulfate, Ethanesulfonate, Methanesulfonate, 1,5-Naphthalenedisulfonate, Toluene 4-sulfonate, alkoxy-naphthalene sulfonates and sulfosuccinate salts having the structure as shown below:

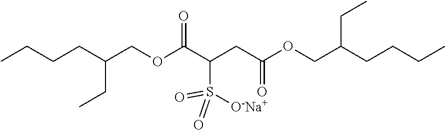

The above structure is known as sodium docusate which is an stool softener when administered at dosages of 50-100 mg. However in the present invention when docusate is used the amount are below the pharmacological effect level.

It should be noted that the metal counterion for the above sulfonate salts can be sodium, potassium, calcium, magnesium, lithium. Ammonium salts could also be used as well as amine salts. Additionally the acid forms of all of the above could be used in the practice of the invention.

Within the context of the foregoing description of the invention all capsules manufactured according to the invention include an organic sulfonate in the gelatin composition used to make the capsules that accelerates the drying rate of the capsules.

The present invention provides an innovative and efficient system for the manufacture of capsules with two or more internal components. Although the internal components may be incompatible the invention is also intended to provide internal components that are compatible but are intended to be released at different intervals.

The present invention provides an advanced drug delivery system that places different pharmaceuticals forms in a single dosage combination. The invention allows delivering incompatible pharmaceutical actives in the form of solid, liquid, microgranules, gels, hard shell or soft gel capsules within an outer softgel capsule.

The novel dosage system allows for combining different therapeutic entities that have never been combined before, via oral, ovules, or suppositories.

For the multi-drugs regimen patients and due to the incompatibility of some actives that can not be combined in a single dose, the instant invention offers a universe of possibilities for current and future new drugs combinations and supplies different releasing delivery.

In the present invention, existing and proven delivery systems are combined in a highly reliable, easy to use and affordable manufacture that give the resulting dosage form unique characteristics to deliver single or multiple APIs regardless of physical-chemical compatibility and/or stability liabilities.

For the multi-drugs regimen patients this delivery system is a viable alternative; due to the manufacturing of IR plus MR combinations in tablets and hard-gelatin capsules while enhancing dosing accuracy and by-passing dissolution barriers and coating issues. It allows the formulation of combination products, highly needed to assure patient compliance and allow synergic clinical effects in a safe and stable dosage form.

Some of the most important advantages are:

Fast and sustained release in a single dose.

Gastric or intestinal release in the same dose.

Fewer intakes to be administered.

Simplicity of regimen reduces mistakes.

Impossible to be falsified.

Reduces number of Rx's prescribed by Physician.

Smaller number of presentations to maintain.

The invention further provides soft-gelatin capsules as a immediate-release (IR) delivery system, that upon rupture, it releases immediate or modified release (MR) tablets, capsules, softgels, granules and/or microgranules. Compatible and/or incompatible pharmaceutical active ingredients, and/or blends of IR and MR dosage forms of the same or different active pharmaceutical ingredients (APIs) can be dosed simultaneously in a single capsule. These capsules may be designed to be administered orally, vaginally or rectally, as needed.

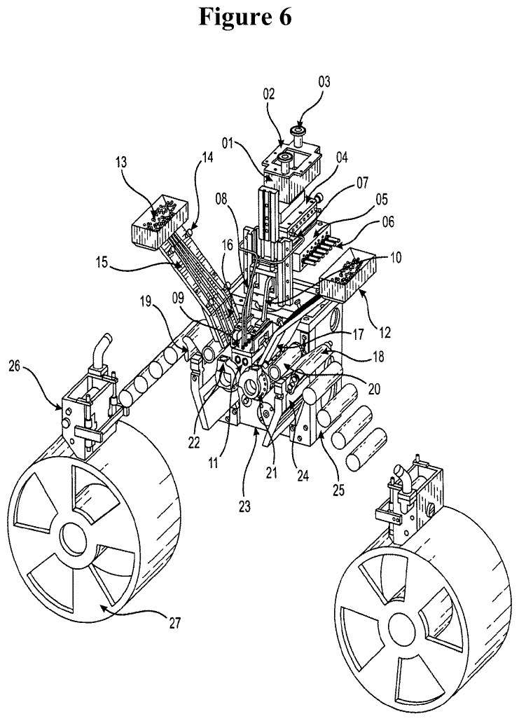

Referring in detail to the apparatus shown in FIG. 6, reference numeral 1 illustrates a medicine hopper having a cover 2 and a medicine feeder 3 connected with a clamp. The apparatus further includes a medicine distributor system 4, pump 5 to pump medicine and further includes plonger 6. The apparatus also includes a fitting distributor connection 7, medicine tubing/hoses 8, a segment coupling connection 9, a support segment 10, and wedge segment 11.

The apparatus has lateral hoppers 12 and 13 containing smaller capsules or other solid dosage forms that are intended to be encapsulated by the soft gels being formed in the rotary dies. The lateral hopper dispensing system includes acrylic or other material knob fasteners 14 and acrylic substrate 15 having guide channels/tracks 16 for the smaller capsules or other smaller solid dosage forms such pellets or minitablets, etc. The lateral dispensing system of the invention includes a grasping claw 17 for dispensing the smaller capsules coming through channels/track 16. The apparatus further includes the conventional aspects of making softgel capsules which includes a gelatin film 18, guiding rollers 19, tensioner 20, rotary mold 21, a vacuum system 22, capsule exit 23 after the capsule is formed, a yoke support arm 24, housing 25, spreader gel dispensing boxes and casting drum 27.

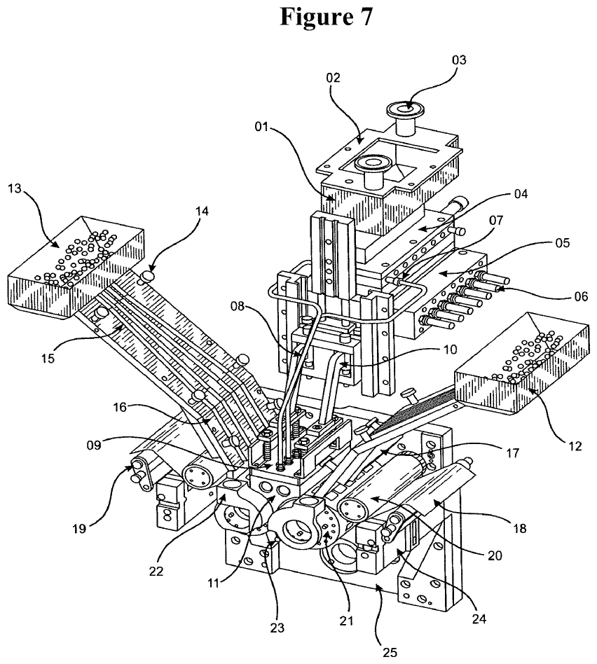

FIG. 7 illustrates the apparatus of FIG. 6 without the spreader gel dispensing boxes and casting drums. The reference numerals in FIG. 7 are identical as those in FIG. 6.

The film-forming materials of the invention comprise at least one component selected from the group consisting of gelatin, starch, carrageenans, gums or synthetic materials such as hydroxypropyl-methylcellulose (HPMC), other hydroxyalkylated celluloses and the like. The film-forming material typically has an aqueous base and is considered to be ingestible. As used herein, the term “ingestible” is used to indicate a film-forming material that dissolves under conditions simulating the human digestion tract or water.

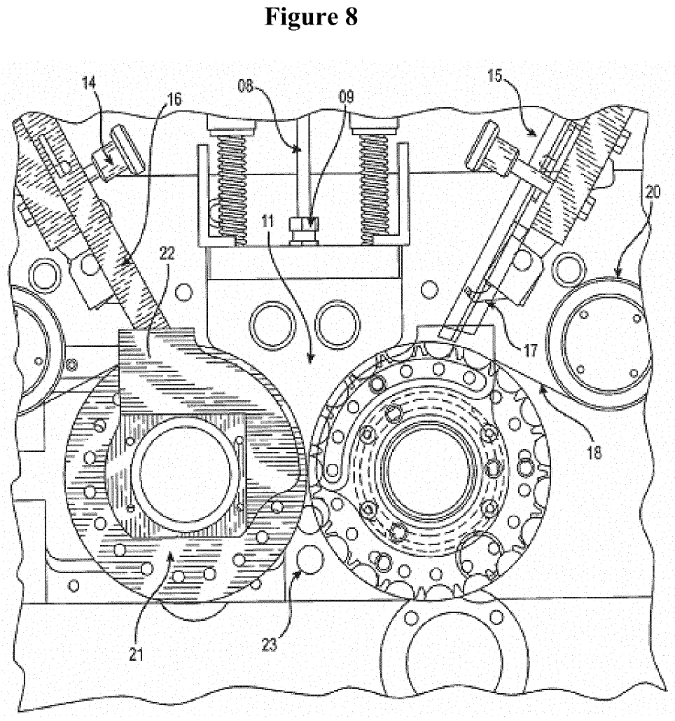

FIG. 8 shows the dispensing and feeding of solid dosage forms or capsules that come from hoppers 12 and 13 (not shown-See FIGS. 6 and 7) controlled by grasping claw 17 with volume capacity for accurate dosing fixed within the capsule. The smaller dosage form or smaller capsules is fed through guide channels 16 and deposited inside a half pocket as the softgel capsule is being formed in rotary die 21. The grasping claw 17 releases each capsule into each packet as the rotary die moves. The final capsule is also filled with additional pharmaceutical actives in liquid form injection tubing 8. After filling the formed capsule 23 falls-through to a conveyor belt and then transported for drying.

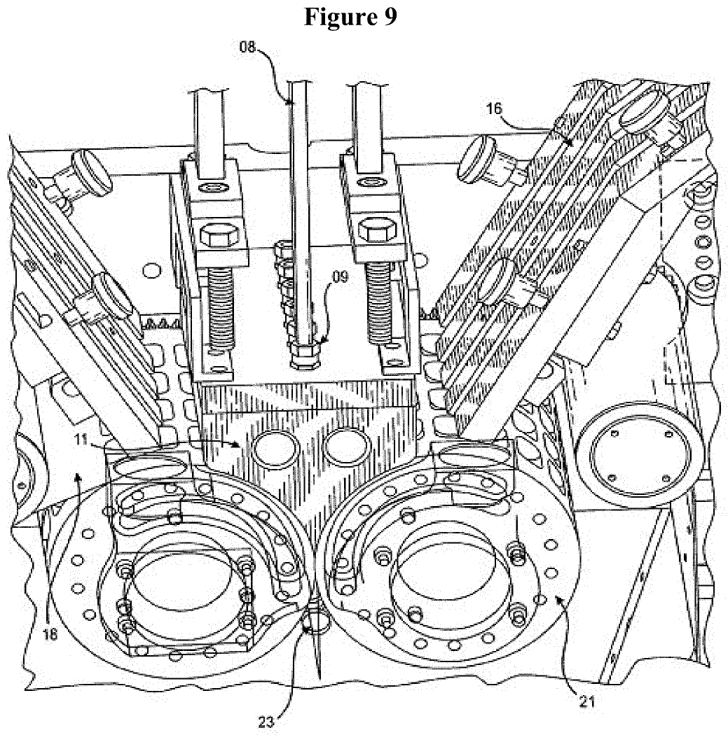

FIG. 9 further illustrates in more details the feeding of solid dosage forms or capsules into the rotary molding process for making softgel capsules containing inetrnally other dosage forms such as smaller capsules, pellets, small tablets, etc. The feeding of the internal capsule is made by an independent dispenser having guide channels 16 so that as capsules are deposited in the pocket of the rotary die/mold 21, the wedge segment 11 is used to simultaneously dispense a liquid medicine product to fill the capsule. As is well known gelatin film 18 is used to form the softgel pocket in the rotary die/mold 21.

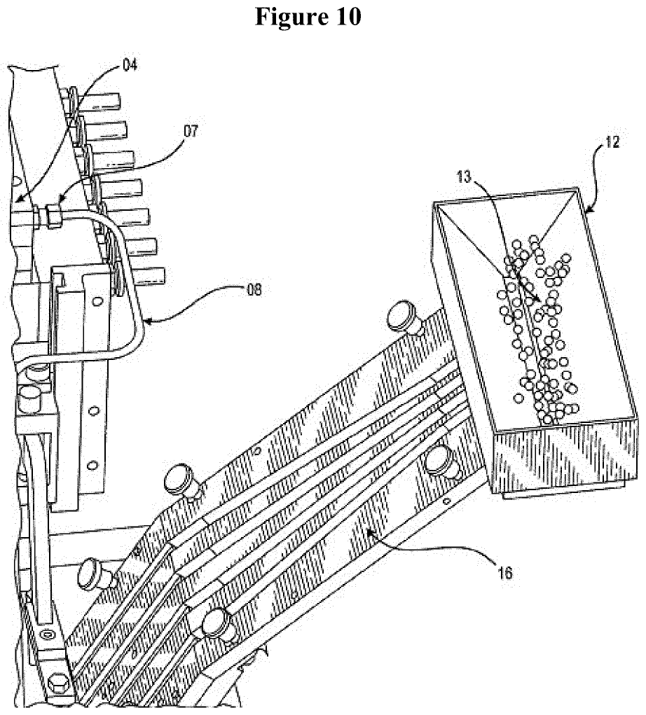

FIG. 10 shows one of the lateral hoppers having smaller solid dosage forms or smaller capsules to be filled inside another softgel capsule. The hopper 12 having capsules 13, are released from the hopper and deposited and guided through guidechannels 16 which in turn leads to the pocket in the rotary mold that is in a tangential position.

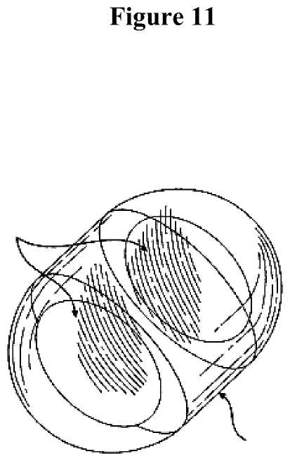

FIG. 11 illustrates a finished capsule of the invention. One or more smaller capsules may be encapsulated in any way into another immersed in a liquid or solution containing a pharmaceutical active ingredient.

The resulting products of the invention include softgel capsules having incorporated therein another solid dosage form selected from the group consisting: (a) one capsule contains an omega oil and the other solid dosage form is a capsule having a statin; (b) one capsule contains a non-steroidal antiinflammatory and the other solid dosage form contains and antihistamine; and (c) one capsule contains and omega oil and the other solid dosage form contains a salicylate.

Typically the omega oil is an omega-3 oil and the statin is selected from the group consisting of mevastatin, lovastatin, pravastatin, fluvastatin, simvastatin, rosuvastatin, cerivastatin and atorvastatin and derivatives and analogs thereof.

The non-steroidal antiinflammatory acid is selected from the group consisting of: ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid and tolfenamic acid, diflunisal, flufenisal and piroxicam.

The antihistamine is selected from the group consisting of: diphenhydramine, loratadine, cetirizine, fexofenadine, hydroxyzine, cyproheptadine, chlorphenamine, clemastine and desloratadine.

The salicylate is typically acetylsalicylic acid.

The present invention provides delivery systems which are combined in a highly reliable, easy to use and affordable manufacture that give the resulting dosage form unique characteristics to deliver single or multiple APIs regardless of physical-chemical compatibility and/or stability labilities. The soft-gelatin delivery system can be filled with hydrophilic or lipophilic media to suspend various IR and/or MR dosage forms in drug solutions or plain liquid phases.

The delivery system of the invention is a viable alternative to the manufacturing of IR plus MR combinations in tablets and hard-gelatin capsules while enhancing dosing accuracy and by-passing dissolution barriers and coating issues. It also solves compatibility and stability issues for multivitamins, cold remedies, nutraceuticals and multiple other OTC medications. The invention also allows the formulation of combination products, highly needed to assure patient compliance and allow synergistic clinical effects in a safe and stable dosage form.

The invention also allows for ease of identification by color coding the shell, fill and/or contents minimizing counterfeiting risks.

The shell of the soft gelatin capsule may be formed from plasticized gelatin or other functional polymeric materials that are typically used for encapsulation of liquids, fluids, pastes or other fill materials.

The outer shell of the soft gelatin capsule may be coated with one or more coatings, including but not limited to, immediate release coatings, protective coatings, enteric or delayed release coatings, sustained release coating, barrier coatings, and combinations thereof. The one or more coatings on the outer shell of the soft gelatin capsule may be useful to provide controlled release of the soft gelatin capsule, protect the soft gelatin shell from degradation, or deliver one or more active ingredients which may be the same or different as those in the liquid phase and solid dosage form. Alternatively, additives such as pectin or synthetic polymers may be incorporated into the soft gelatin capsule shell to slow the dissolution on ingestion. Such coatings or additives to the soft gelatin shell phase are well described in the literature and known to those experts in the field. The one or more coatings outer shell of the soft gelatin capsule may be applied by any conventional technique, including but not limited to, pan coating, fluid bed coating or spray coating.

The liquid fill phase of the soft gelatin capsule may comprise one or more liquids for carrying the solid dosage form that are compatible with the soft gelatin shell and do not interfere with or degrade the solid dosage form. The liquid fill phase may consist of one or more combinations of fluids that may be broadly categorized as hydrophilic or lipophilic.

A lipophilic liquid fill phase may be an oil form of an active ingredient, an active ingredient or multiple active ingredient preparation that may be solutions, suspensions, emulsions, micro-emulsions, self-emulsifying systems, and other liquids that will be known to those who are expert in the field of soft gelatin capsules formulation. Examples of useful oils include omega-3 fatty acids, vegetable oils, mineral oils or other food grade oil. Vegetable oils may include castor bean oil, coconut oil, peanut oil, palm kernel oil, canola oil, avocado oil, evening primrose oil, rice bran oil, borage oil, sunflower oil, soybean oil, palm oil, corn oil and safflower oil. Preferred oils are omega-3 fatty acids triglycerides or ethyl esters. Examples of omega-3 fatty acids include alpha-linolenic acid, eicosapentaenoic acid and docosahexaenoic acid.

Hydrophilic liquid fill phases are typically based on polyethylene glycols commonly referred to as PEG 400 or PEG 600 with lesser amounts of glycerol, propylene glycol and water in proportions designed to affect the moisture balance between the liquid fill phase and the capsule shell. Such combinations are commonly known to experts in the field.

Other hydrophilic materials used to a lesser extent that are, but not limited to, methoxypolyethylene glycols, diethyleneglycol monoethyl ether tetrahyrofurfuryl alcohol polyethylene glycol, propylene carbonate, n-methyl-2-pyrrolidone, polyoxyethylene-poly-oxypropylene copolymers benzyl alcohol and ethyl alcohol.

The fill formulation may be prepared using established procedures employed for manufacture of pharmaceutical solutions, suspensions and semi-solids, and recognized by those experts in the field of soft gelatin formulation.

Individual or multiple liquid phases may be introduced into the capsule by means of a single, dual or multiple wedge design that facilitates in-situ capsule filling of multiple phases.

The liquid fill phase may include different liquid phases which are layered side-by-side in the soft gelatin capsule. Each layered phase may incorporate an active ingredient or multiple active ingredients.

The fill materials may also include excipients known in the art of soft gelatin encapsulation such as dispersants, surfactants, plasticizers, flavoring agents, opacifying agents, preservatives, embrittlement inhibiting agents, colorants, dyes and pigments, and disintegrants.

The solid phase may be in the form of preformed tablets, caplets, capsules, slugs of solid materials, granules or other solid dosage forms. Preferably, the solid phase is comprised of a preformed solid dosage form. More preferably, the preformed solid dosage form is a pharmaceutical finished dosage form, which is a dosage form suitable for administration to human or animal subject, the pharmaceutical characteristics of which are acceptable and may be approved by regulatory authorities previously or subject to assessment by regulatory agencies.

The shape and size of the solid dosage form can vary in accordance with the invention. The shape of the capsule may be, but is not limited to, round, oval, oblong, or a non-standard shape. The solid dosage form is sized to be less than the total internal volume of the soft gelatin capsule.

The solid dosage form may be coated with one or more coatings, including but not limited to, immediate release coatings, protective coatings, enteric or delayed release coatings, sustained release coatings, barrier coatings, moisture shield coatings and combinations thereof.

The one or more coatings on the solid dosage form are useful to provide controlled release of an active ingredient in the solid dosage form, protect the solid dosage form from interactions with the liquid fill phase, or deliver one or more active ingredients. Preferably, the solid dosage form is film coated. The one or more coatings on the solid dosage form may be applied by any conventional coating technique recognized in the pharmaceutical industry, including but not limited to, pan coating, fluid bed coating or spray coating. Optionally, the coated or uncoated solid dosage form may be enrobed in gelatin film according to well known conventional tablet enrobing techniques.

Typical immediate release coating films are hydro-alcoholic film coatings or cellulose film coating systems as used in various pharmaceutical solid oral dosage forms. Typical coating systems may be aqueous, alcohol or organic solvent based or combinations containing hydroxy-propyl-methyl cellulose and derivatives, and polyvinyl alcohol and derivatives. Examples of film coated tablets include: Amoxicillin, Azithromycin, Atenolol, Amlodipine, Acelofenac, Amtriptyline, Ampicillin HCl, Ciprofloxacin, Cefadroxil HCl, Celecoxib, Cimitidine, Calcium Tablets, Certizine HCl, Clarithromycin, Chloroquine Phosphate, Erythromycin estolate, Erythromycin striate, Enalpril Maleate, Elctronxib, Ferrous, fumarate, Famotidine, Flupenthixol, Fluoxetine Felodipine, Gatifloxacin, Gliclazide, Ibuprofen, Indapamide, Ketorolac, Ketoprofen, Levofixation, Levocetrinzie, Losartan, Potassium, Levamisole, Metormin, Methylopa, Metra+Tetraozole, Metronidozole, Methyl, Corn blamine, Mefenamic acid, Metropralal Nifedipne, Norfloxacin, Nifedopine, Norfloxacin, Norflax+Tindazole, Oflaxacin, Oflaxacin+Omidazole, Olazzapine, Orridazole, Oflexacin+Omidazole Paracetamol, Pravastain, Prmethazine, Quinine, sulphate, Primaquine, Ramipril, Tindazole, Tiri+Doxicycline, Tiri+Tetracyline, Valdecoxib, Verapamil, herbal and Neutraceuticals.

Typical protective coatings may include, but is not limited to, antioxidants, chelating agents, colours or dyes.

Typical enteric coatings of the solid dosage form may consist of, but not limited to, one or more of the following recognized coating agents: methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate/alginic acid and stearic acid. Examples of enteric coated tablets include: Aspirin and Clopidogrel combination, Aspirin, Bisacodyl, Diclofenac-sodium, Doxylamine succinate, Esomeprazole, Garlic Tablets, Lansoprazole, Omeparazole, Pantoprazole, Pentoxyfilline, Pancreatin, Rabeprazole, Serratiopeptidase, and Sodium Valproate.

Sustained release tablets may be film coated, enteric coated, or polymer matrix formulated. Sustained release film coatings may include, but is not limited to, a water insoluble material such as a wax or wax-like substance, fatty alcohols, shellac, zein, hydrogenated vegetable oils, water insoluble celluloses, polymers of acrylic and/or methacrylic acid, and any other slowly digestible or dispersible solids known in the art. Examples of sustained release dosage forms include: Acetazolamide Pellets, Aminophylline, Amitriptyline Pellets, Captoprill, Diclofenac Sodium, Diltiazem, Gliclazide, Iron, Levodopa, Lithium Carbonate, Metformin, Methyldopa, Nifedipine, Salbutamol Sulphate, Theophylline, Verapamil HCL, vitamin supplements, mineral supplements, and vitamins with Zinc.

Moisture shield coatings provide moisture barriers for moisture sensitive or hygroscopic drugs. Such coatings may be applied to solid dosage forms to protect the solid dosage form moisture resulting from, for example, the soft gelatin encapsulation process of which utilizes water as a processing aid and primary plasticizer of the gelatin or functional polymer capsule shell system. Examples of dosage forms incorporating moisture shield coatings include, but are not limited to: Amitriyptyline HCl, Amoxycillin and Clavulanic Acid combination, Atorvastatin and Calcium combination, Calcium Tablets, Clopidogrel, Ethambutol, Glucosamine and Chondritin combination, certain Herbal products, Multivitamins, Ranitidine HCl, Rifampicin and other hygroscopic drugs.

The active ingredients introduced in the liquid phase and solid dosage form of the multi phase soft gelatin capsules of the present invention may comprise APIs, nutritional supplements, substances used for therapeutic purposes, functional excipients or combinations of active ingredients and functional excipients that control or otherwise affect the release of the active ingredient(s) into the gastrointestinal tract or site of absorption. Each of the liquid phase and solid dosage form may contain one or more active ingredient(s). The active ingredient(s) in the liquid phase and the active ingredient(s) in the solid dosage form may be the same or different.

The present invention contemplates the use of any active ingredients known in the art. It is well within the knowledge of a skilled person in the art to select a particular combination of active ingredients or medicaments. In some embodiments, active ingredients may include, but are not limited to, the following: APIs, nutraceuticals, nutritional supplements, therapeutic substances, and functional excipients.

APIs may include, but are not limited to, the following: analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, anti-protozoal agents, anti-pyretics anti-thyroid agents, anti-tussives, anxiolytics, sedatives, hypnotics, neuroleptics, neuroprotective agents, beta-blockers, cardiac inotropic agents, cell adhesion inhibitors, corticosteroids, cytokine receptor activity modulators, diuretics, anti-Parkinson's agents, gastrointestinal agents, histamine H-receptor antagonists, HMG-CoA reductase inhibitors, keratolytics, lipid regulating agents, muscle relaxants, nitrates and other anti-anginal agents, non-steroid anti-asthma agents, nutritional agents, opioid analgesics, sex hormones, stimulants, and anti-erectile dysfunction agents.

Nutraceuticals may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin B12), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Nutritional supplements may include vitamins, minerals, fiber, fatty acids, amino acids, herbal supplements or a combination thereof.

Vitamins may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Herbal supplements may include, but are not limited to, the following: arnica, bilberry, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Preferred nutritional supplements include, but are not limited to, the following: B vitamins and Vitamin B complex, beta-carotene, calcium, collagen, Co-Q-10, cranberry, echinacea, flax seed oil, folic acid, garlic, ginger, ginseng, glucosamine, chondroitin, green tea, iron, lecithin, lutein, lycopene, magnesium, melatonin, milk thistle, niacin, Omega-3 oils, potassium, probiotics, saw palmetto, selenium, St John's wort, tocopherols, valerian, vitamin A, vitamin B12, vitamin C, vitamin D, vitamin E, zinc and combinations thereof. Preferred nutritional supplement combinations include: Co-Q-10 and Omega-3 oils; echinacea, garlic and ginger; glucosamine and chondroitin; vitamin D and calcium; vitamin D, calcium and magnesium; vitamin D, calcium, magnesium and zinc; and vitamin E and other tocopherols.

Soft gelatin capsules containing solid and liquid phases according to the invention provide a number of significant benefits for the administration of active ingredients.

The multi phase soft gelatin dosage form of the present invention can be used to deliver two or more active ingredients that otherwise would interact with each other. One or more active ingredients are dissolved in the liquid fill phase and the other active ingredient(s) in the solid dosage form.

Another use of the present invention is to provide effective control of the release of single or multiple APIs introduced in the solid dosage form and liquid phase. The liquid phase provides the capability of immediate release of the API in the liquid phase by virtue of a solution, pre-dispersed or self-emulsifying formulation. The solid phase may be coated to provide delayed release of the API in the solid phase.

When two or more capsules and or tablets are prescribed, their combination into one dosage form provides patient benefits and administration advantages of convenience and costs.

Analgesics such as ibuprofen and acetaminophen differ in their mode of action and related therapeutic effects such that combined administration provides improved analgesia and safety. The present invention provides the capability to engineer combinations of solid dose components of acetaminophen, ibuprofen or other combinations as individual solid forms or combined in one solid form with a liquid phase that may contain either or other active ingredients.

The present invention may be useful as a polypill. A polypill is a medication that contains a combination of active ingredients, reducing the number of tablets or capsules that need to be taken. Combined medications in the form of a polypill are useful for the treatment of cardiovascular disease and diabetes.

The present invention may reduce problems, such as time and expense, associated with combining two or more APIs into one dosage form. New combinations in one dosage form require development of new formulations and require pharmaceutical and clinical studies to demonstrate safety, efficacy and potency. The present invention provides the capability to incorporate more than one API into a unit dosage form utilizing established forms of the API or APIs that may be in liquid or solid phases. The present invention allows retention of the established tablet, caplet or capsule form of an API. This provides the capability to retain the pharmaceutical characteristics of the solid dosage form in combination with a liquid or fluid phase. Key characteristics include: physical and chemical stability, API release profile of the tablet or caplet, bioavailability and clinical performance. Retention of the established clinical performance by incorporation of the original solid may avoid the need to conduct extensive Phase III clinical studies that would otherwise be required with a new formulation.

By combining a number of established, off patent or generic medications, the present invention may be useful to treat cardiovascular conditions and provides the prospect of low cost treatment.

The present invention may be useful for the combined administration of unit doses of HMG-CoA inhibitors (statins) and Omega-3 fatty acids. The statins may be in the form of tablets or capsules containing single ingredient HMG-CoA inhibitors, or statins in combination with other active ingredients. Commercially available statins include LIPITOR™ or TORVAST™ (atorvastatin calcium) sold by Pfizer, LESCOL™ or LESCOL XL™ (fluvastatin sodium) sold by Novartis, MEVACOR™, ALTOCOR™ or ALTOPREV™ (lovastatin) sold by Merck, LIVALO™ or PITAVA™ (pitavastatin) sold by Kowa, PRAVACHOL™ SELEKTINE™ or LIPOSTAT™ (pravastatin sodium) sold by Bristol Myers Squibb, CRESTOR™ (rosuvastatin calcium) sold by AstraZeneca, ZOCOR™ or LIPEX™ (simvastatin) sold by Merck. Examples of established tablet dosage forms containing two or more active ingredients include VYORTIN™ (simvastatin+exetimibe) sold by Merck, ADVICOR™ (lovatatin+niacin) sold by Merck, CADUET™ (atorvastatin calcium and amlodipine besylate) sold by Pfizer, and SIIVICOR™ (simvastatin+niacin) sold by Merck. The Omega-3 fatty acids may be in the form of ethyl esters or tri-glycerides.

The present invention may permit the use of a smaller dosage form than those commercially sold. Encapsulation of a solid dosage form in a soft gelatin capsule provides protection against dissolution of the solid dosage form prior to reaching the intended target site. Thus, an encapsulated solid dosage form may not need to be as durable as compared to commercially available solid dosage forms. The present invention may reduce the need for excipients which function to prevent early dissolution of the solid dosage form when ingested, permitting the use of a smaller and cheaper solid dosage form.

The following are examples of the benefits of the present invention for the administration of combinations of medicines that may be in the form of a solid dosage form and liquid fill phase in a soft gelatin capsule.

The following examples further describe and demonstrate embodiments within the scope of the present invention. The examples are given solely for the purpose of illustration and are not to be construed as limitations of the present invention, as many variations thereof are possible without departing from the spirit and scope of the invention.

### EXAMPLES

The following examples are intended to demonstrate the usefulness of preferred embodiments of the present invention and should not be considered to limit its scope or applicability in any way.

### Example 1

### Soft Gelatin Capsule Containing a Placebo Fill

A soft gelatin mixture is prepared from the following ingredients.

| INGREDIENT | WEIGHT % |
|---|---|
| Gelatin | 48.00 |
| Glycerin | 14.00 |
| Organic Sulfonate | 0.02 mg to 20 mg |
| Water | QS 100 |

The above ingredients are combined in a suitable vessel and heated with mixing at about 65° C. to form a uniform solution. Using standard encapsulation methodology, the resulting solution is used to prepare soft gelatin capsules. The resulting capsules are dried in trays and/or tumbler and the time of drying is compared to the capsules not including organic sulfonate salts. The capsules containing organic sulfonate salts such as sodium docusate enhance the drying rates when compared to capsules not containing organic sulfonate salts.

### Example 2

Using carbohydrate gums suitable for making softgel capsules similar formulations are manufactured as in Example 1.

### Example 3

### Experimental Objectives

1. To evaluate the effect of adding sodium docusate 100% USP in the gelatin masses at different concentration levels on the drying time of a prototype of soft gelatin capsule containing a hydrophilic fill content.

2. To evaluate the effect of the plasticizers on the drying time of a prototype of soft gelatin capsule added of sodium docusate 100% USP

Samples

The fill content contains polyethyleneglycol as main component, propyleneglycol not exceeding 10% and a water soluble color to test the dissolution in the capsules. Water was not added to the fill content formulation in any of the samples. The target dose was 285 mg/capsule for a 5.5 oval capsule size.

The gelatin formulation contains gelatin 150 bloom, type B at standard use level and sorbitol-glycerol (S-G) mixtures as plasticizer. The ratio S/G ranged from 0.7-1.55 and the ratio gelatin plasticizer remained constant. SD content was tested in each sample by means of the assay method under docusate sodium capsules (USP 41).

TABLE 1
Tested Systems

| | Sorbitol/Glycerol | | |
|---|---|---|---|
| Sample | ratio | Fill content | SD mg/capsule |
| blank | 0.67 | Blank (w/o SD) | N.A |
| S/G low level | 0.67 | SD 1.5% + fill | 4 |
| S/G high level | 1.5 | SD 1.5% + fill | 5.1 |
| blank | 1.5 | Blank (w/o SD) | N.A |
| SD low level | 1.5 | SD 2.0% | 2.7 |

N.A. Not apply

### Results

### Example 3A

### Effect of the Sorbitol/Glycerol Ratio in the Drying Time of a Hydrophilic SGC Prototype Added with Sodium Docusate in the Gelatin Formulation

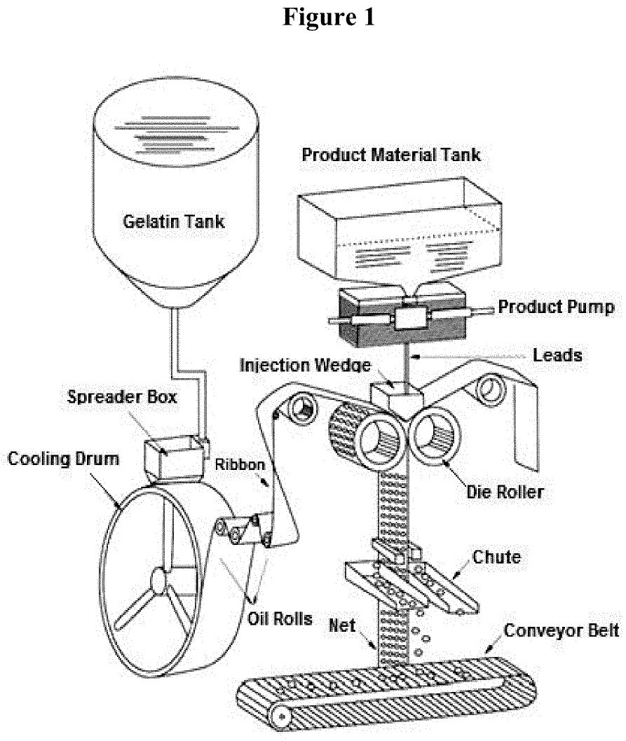

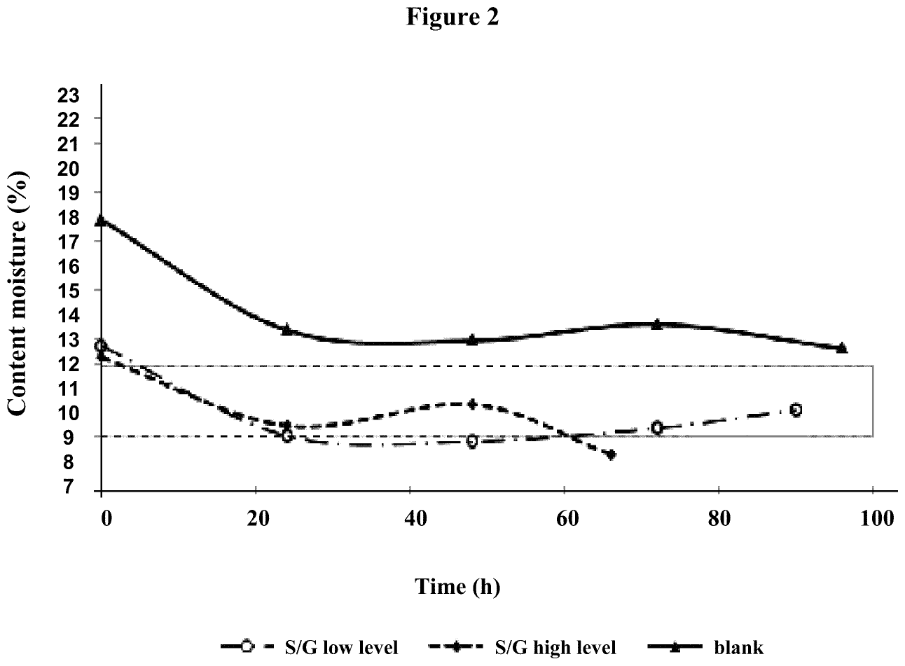

Samples in FIG. 2 contain the same amount of SD 100% USP added in the shell (1.5%) and SD in polyethyleneglycol 50% added in the fill, but low and high levels of the ratio sorbitol/glycerol as plasticizer. Drying curves of the samples were compared in FIG. 1 to evaluate the effect of the ratio S/G in the SD migration from the fill to the shell and the effect on drying. It was observed that the drying of the sample S/G low level was faster in correspondence to a high assay value of sodium docusate in the shell (27% higher). That explains that sorbitol promotes the migration of sodium docusate to the shell. Both samples with docusate addition met the specification for water content in the fill at 24 h compared to the blank sample that did not met it during the tested period (96 h).

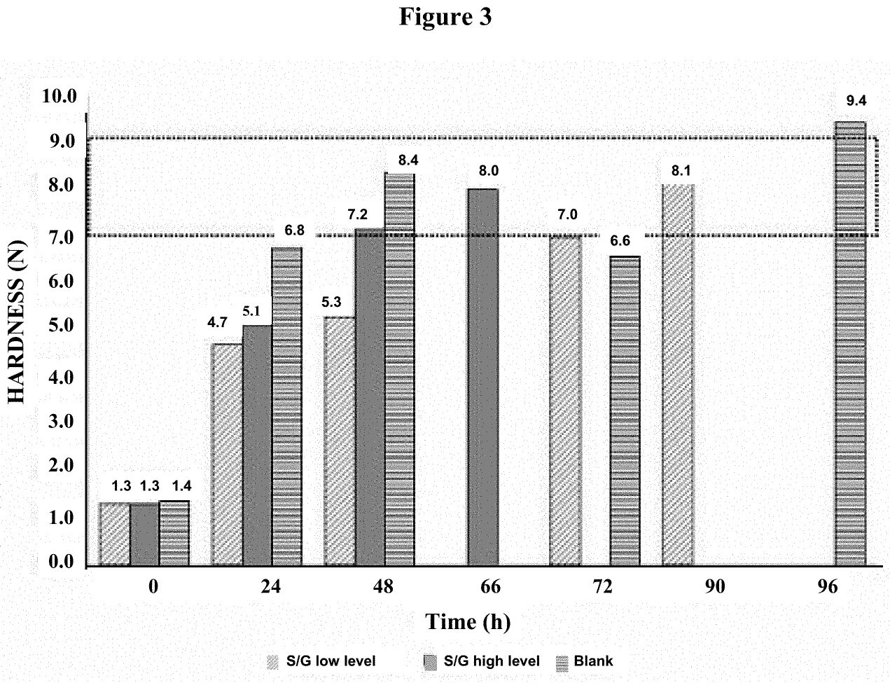

The acceptance criteria for hardness (7-9N) was met for sample S/G low level and S/G high level at 72 and 48 h, respectively (FIG. 3). The more the glycerol content in the shell formulation (sample S/G low level), the longer the time to meet the hardness specification. Low hardness values are also related with the polyethyleneglycol in the fill content that migrates to the shell and acts as a plasticizer. It was also observed that the samples showed clumping at accelerated conditions. For this reason, the level of PEG in the fill content should be limited to 20% to avoid over plasticizing the shell and the SD should be added only in the shell formulation.

### Example 3B

### Effect of the Concentration of Sodium Docusate (SD) 100% USP in the Drying Time of a Hydrophilic SGC Prototype

To evaluate the effect of SD concentration, SGC were obtained at two SD levels using as plasticizer the mixture S-G at its high tested level.

As concluding results, the lowest drying time (48 h) for the prototype SGC was obtained at 2.0% and 2.5% of sodium docusate in the gelatin mass. The drying curves were compared through the similarity facto (F1) and SD samples were found to be comparable. In contrast, the moisture in the shell and the fill was significant different comparing the docusate-added samples with the blank sample (F1>15). In the FIG. 3, the drying curves for water content in the fill are shown for the blank samples and the gelatin masses added with docusate. The specification for the water content in the fill is 9-12% was met at 48 h hours for both samples added with docusate and it was not met by the blank sample in the tested period (168 h). The drying process reduced the water content in the fill from 19-21% after encapsulation to 10-11.8% at 48 h in the capsules added with docusate. The blank at the same time registered 13.7% water in the fill content. The water content in the shell decreased from 13.7-16.5% to 13.8-14.1% at 48 h compared to 14.6% for the blank at the same drying time.

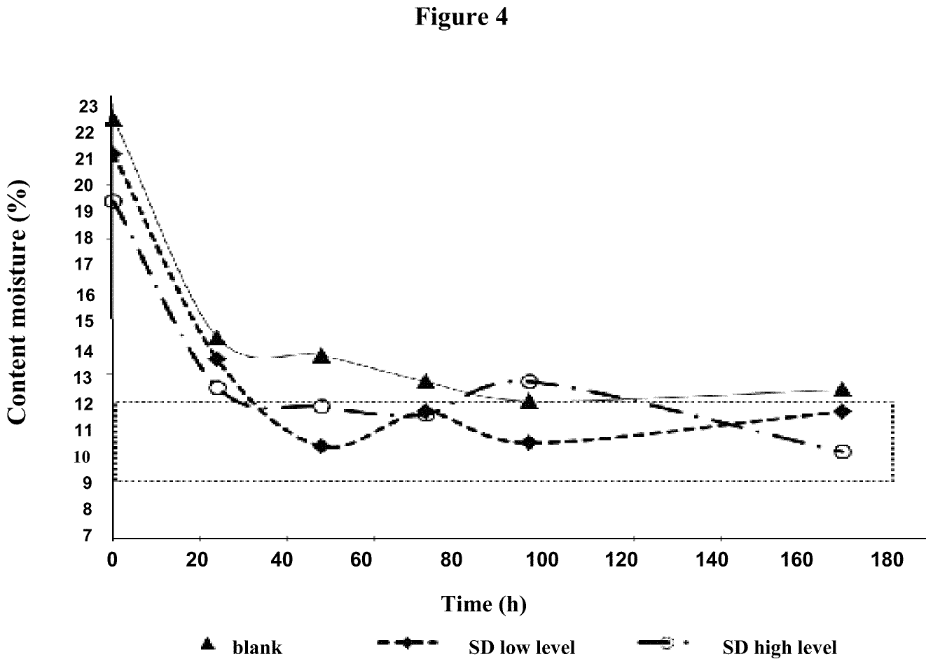

As it can be seen in the FIG. 4, the moisture content in the fill for the samples with SD reaches a steady state from 48 h (RSD 0.7-1.1).

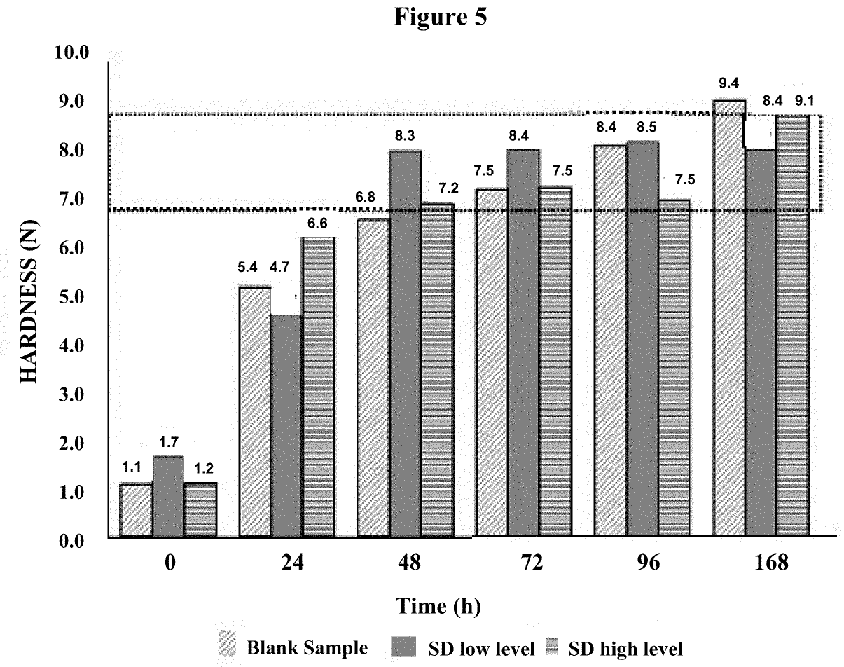

Samples were also tested for hardness (FIG. 5) and mechanical resistance (burst test) to determine the effect of SD on these physical properties. The specification for hardness (7-9 N) was met for the samples with docusate at 48 h. For the low level of SD, the hardness remains stable (RSD 0.08) in the tested period (up to 168 h), meanwhile for high level of SD and blank samples increase up to 9.1 and 9.4N, respectively.

The results for mechanical resistance showed the highest values for the blank sample and similar values for docusate samples from 96 h (95-102 N). In spite of the seal is about a 50% weaker in the docusate samples compared to the blank sample, the capsules did not show leakers during the predrying or the drying step or in their handling.

### CONCLUSIONS

The selected plasticizer system was sorbitol-glycerol in the high tested S/G range. It showed migration in less extent between the fill content and the shell. Capsules obtained using this plasticizer level met the hardness specification at 48 h.

The effect of the addition of SD to gelatin masses on reducing the drying time of SGC was demonstrated through the drying curves of the SD samples, that were different compared to the blank sample (F1>15). The blank sample did not met the specification for the moisture content in the fill in the tested period while the SD samples met the specification at 48 h, reached the steady state from this time and met the hardness criteria at 48 h.

In regards the SD concentration, there was not difference between the low and high SD level when compared through the similarity factor (F1).

The contents of all references cited in the instant specifications and all cited references in each of those references are incorporated in their entirety by reference herein as if those references were denoted in the text.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A softgel capsule having incorporated in the shell an amount of an organic sulfonate salt which is effective to accelerate the drying rate of said capsule but not effective to provide a pharmacological effect.

2. The softgel capsule of claim 1, wherein said organic sulfonate salt is an aromatic sulfonate salt.

3. The softgel capsule of claim 1, wherein said organic sulfonate salt is an aliphatic sulfonate salt.

4. The softgel capsule of claim 2, wherein said aromatic sulfonate salt is selected from the group consisting of unsubstituted and substituted benzene sulfonates and unsubstituted and substituted naphthalene sulfonates.

5. The softgel capsule of claim 4, wherein said salts are selected from the group of alkaline metal salts and alkaline earth salts.

6. The softgel capsule of claim 3, wherein said aliphatic sulfonate salt is derived from an ester of an aliphatic dibasic acid having the formula 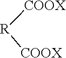
in which R is an aliphatic carbon chain containing at least one sulphonic group, but free from other substituents, and X is an alcohol or phenol radical not connected by a carbon to carbon bond with R.

7. The softgel capsule of claim 3, wherein said aliphatic sulfonate salt is derived from an ester of an aliphatic dibasic acid having the formula 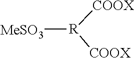
in which R is an aliphatic carbon chain containing at least one sulphonic group but free from other substituents, and X is hydrogen or an alcohol or phenol radical not connected by a carbon to carbon bond with R, at least one X being such an alcohol or phenol radical and Me is hydrogen or a base.

8. The softgel capsule of claim 3, wherein said aliphatic sulfonate salt is derived from an ester of an aliphatic dibasic acid having the formula 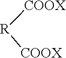
in which R is an aliphatic carbon chain containing at least one sulphonic group, but free from other substituents, and X is an alcohol or phenol radical not connected by a carbon to carbon bond with R.

9. The softgel capsule of claim 3, wherein said aliphatic sulfonate salt is derived from an ester of an aliphatic dibasic acid having the formula 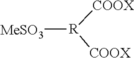
in which R is an aliphatic carbon chain containing at least one suiphonic group, but free from other substituents, X is an alcohol or phenol radical not connected by a carbon to carbon bond with R, and Me is hydrogen or a base.

10. The softgel capsule of claim 3, wherein said aliphatic sulfonate salt is derived from an ester of an aliphatic dibasic acid having the formula 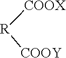
in which R is an aliphatic carbon chain containing at least one sulphonic group and free from mercapto groups and X is an alcohol or phenol radical and Y is a different alcohol or phenol radical.

11. The softgel capsule of claim 3, wherein said aliphatic sulfonate salt is derived from an ester of an aliphatic dibasic acid having the formula 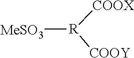
MeSO₃—R in which R is a carbon chain free from mercapto groups, Me is a base, X is an alcohol or phenol radical and Y is a different alcohol or phenol radical.

12. The softgel capsule of claim 6, wherein said organic sulfonate salt is sodium docusate.

13. A softgel capsule having incorporated therein another smaller tablet or smaller capsule wherein said smaller tablet or smaller capsule has solid or encapsulated active ingredients that are not compatible with another solid or encapsulated active in the softgel capsule and wherein said capsule incorporates a drying accelerator.

14. The capsule of claim 13 wherein said drying accelerator is sodium docusate.

15. In the process of manufacturing softgel capsules of claim 1 by the rotary die process, the improvement which comprises incorporating an organic sulfonate salt in the capsule shell formulation said organic sulfonate being present in effective amounts to accelerate the drying rate of said capsules.

16. The use of an organic sulfonate salt to enhance the drying rate of softgel capsules wherein said sulfonate is ester of an aliphatic dibasic acid having the formula 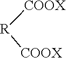
in which R is an aliphatic carbon chain containing at least one sulphonic group, but free from other substituents, and X is an alcohol or phenol radical not connected by a carbon to carbon bond with R.

17. The softgel capsule product of claim 13, wherein said product is selected from the group consisting of:
(a) one softgel capsule contains an omega oil and the other solid form incorporated into the softgel capsule containing the omega oil contains a statin;
(b) one softgel capsule contains a non-steroidal antiinflammatory and the other solid form incorporated into the softgel capsule containing the non-steroidal antiinflammatory contains an antihistamine; and
(c) one softgel capsule contains an omega oil and the other solid form incorporated into the softgel capsule containing the omega oil contains a salicylate.

18. The softgel capsule of claim 17, wherein said omega oil is an omega-3 oil and the statin is selected from the group consisting of mevastatin, lovastatin, pravastatin, fluvastatin, simvastatin, rosuvastatin, cerivastatin and atorvastatin and derivatives and analogs thereof.

19. The softgel capsule of claim 17, wherein said non-steroidal antiinflammatory acid is selected from the group consisting of: ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, indomethacin, sulindac, tolmetin, zomepirac, diclofenac, fenclofenac, alclofenac, ibufenac, isoxepac, furofenac, tiopinac, zidometacin, acemetacin, fentiazac, clidanac, oxpinac, mefenamic acid, meclofenamic acid, flufenamic acid, niflumic acid and tolfenamic acid, diflunisal, flufenisal and piroxicam and said antihistamine is selected from the group consisting of: diphenhydramine, loratadine, cetirizine, fexofenadine, hydroxyzine, cyproheptadine, chlorphenamine, clemastine and desloratadine.

20. The softgel capsule of claim 17, wherein said salicylate is acetylsalicylic acid.
